(19) United States
(12) Patent Application Publication
(10) Pub. No.: US 2018/0264050 A1
(43) Pub. Date: Sep. 20, 2018

(54) (EN) POTENTIATED T-CELL MODULATOR ABLE TO MODULATE IMMUNE RESPONSE, METHOD FOR EXTRACTING, TESTING AND COUNTING A DIALYSABLE LEUCOCYTE EXTRACT FROM SHARK SPLEEN TO PRODUCE SAME, AND THERAPEUTIC USE THEREOF

(71) Applicant: HECTOR MANUEL ZEPEDA LÓPEZ, Naucalpan Estado de México (MX)

(72) Inventor: HECTOR MANUEL ZEPEDA LÓPEZ, Naucalpan Estado de México (MX)

(21) Appl. No.: 15/579,496

(22) PCT Filed: Jun. 4, 2015
(86) PCT No.: PCT/MX2015/000088

§ 371 Date: May 29, 2018

(51) Int. Cl. A61K 35/60; C12N 5/078; A61K 49/00; C12Q 1/06; G01N 33/68; B01D 61/24; B01D 61/14; B01D 61/58

(52) U.S. Cl. CPC A61K 35/60; C12N 5/0634; A61K 49/0008; C12Q 1/06; G01N 33/6863; B01D 2311/02; B01D 61/147; B01D 61/58; B01D 2311/2692; B01D 2311/2676; B01D 61/243

(57) ABSTRACT

The present invention relates to a potentiated T-cell modulator (TCM), with a potency of 10¹² leucocytes/mm³, obtained from a dialysed extract of leucocytes from the spleen of Selachimorpha or sharks that contains peptides equal to or less than 10,000 Da, in powder form. The invention also relates to the use of the TCM to produce a medicine for treating diseases related to the regulation of immune response such as cancer or viral infections.

### TECHNICAL FIELD

The present invention is a method of extracting, checking and counting, of dialyzable extract of leukocytes from leukocyte cells containing polypeptides equal to or less than 10,000 daltons of origin of selacimorphs spleen, commonly known as sharks, to obtain a potentialized T-cell modulator (TCM for its acronym in English) capable of modulating the immune response through the activation of specific molecules involved in the control of innate immunity called “Toll-like Receptors”.

The procedure essentially involves sterilization, extraction of shark spleen, counting and quantification, breaking or separation of components, dialysis, filtration and sterilization, formulation, physical-chemical evaluation and evaluation of biological activity.

The means for obtaining a dialyzable extract of leukocytes containing polypeptides less than or equal to 10,000 Daltons whose source or origin is from cells, tissues or organs of sharks, more specifically shark spleen, of the present invention, is considered novel, since when the prior art was analyzed, methods were found for said extraction of leukocytes, white cells or T cell modulator, by means of leukocytic packages of healthy donors (humans); eggs of reptiles, amphibians, fish and birds; besides colostrum (milk produced by mammal) and crocodile spleen. However no document was found which mentioned the possibility of extracting leukocyte cells from selacimorphs for the extraction of TCM, which are a superorder of chondrichthyes (cartilaginous fish) commonly known as sharks.

### BACKGROUND

In the prior art, there is not any source that provides a concentration of T cell modulator of around 10¹² leukocytes×mm³. Since in said prior art, the T cell modulator concentrations of the extraction media described above, ranges from 10⁴ to 10⁸ leukocytes×mm³ (see FIG. 1).

For example, the patent document WO2002/024746. TRANSFER FACTOR FROM BIRD EGGS. Said document uses a method to obtain a transfer factor from the egg yolk of animals, however, a person skilled in the art knows that the method to extract transfer factor from eggs is very complicated, especially in the separation of lipids. Likewise the units of transfer factor obtained by egg is thousands of times less than the present invention.

Likewise, the document U.S. Pat. No. 4,816,563. PROCESS FOR OBTAINING TRANSFER FACTOR FROM COLOSTRUM, TRANSFER FACTOR SO OBTAINED AND USE THERE OF; uses as a source of extraction of the transfer factor the colostrum of animals, specifically mammals. However, like the previous document, the amount of transfer factor units obtained from colostrum is minimal compared to the present invention, since the TCM found in sharks is 10¹² leukocytes×mm³, against 10⁶ leukocytes×mm³ of the colostrum.

In the same way, the document WO 2005/028622. COMPOSITIONS CONTAINING DIFFERENT TYPES OF TRANSFER FACTOR, METHODS FOR PREPARING COMPOSITIONS AND METHODS OF TREATMENT USING THE COMPOSITIONS. This document combines a composition to produce an immune response mediated by T cells in an individual, which contains the transfer factor of at least two different types of animals. For example, the composition may contain the mammalian transfer factor and a non-mammalian transfer factor. An example of the composition may be a combination of a colostrum-derived product, which includes the mammalian transfer factor, and an egg-derived product, which includes the non-mammalian transfer factor. Like the previous ones, this document presents two problems, eliminating the lipids contained in the egg yolk and the low amount of leukocytes×mm³, which reduces the power to said invention.

In the same way, it was found that the patent MX 9504215 IMPROVED PROCEDURE FOR THE PURIFICATION OF OLIGOPEPTIDES WITH MOLECULAR WEIGHTS OF 1000 TO 10,000 DALTONS, FROM LYMPHOCYTE STRIPS AND ITS PHARMACEUTICAL PRESENTATION, this document refers to an improved procedure for fractionation with high yield, from a set of oligopeptides (from 1000 to 10000 daltons), recovered from the breakdown of leukocytes and possessing biological activity for the regulation of the immune response comprising the following steps: from leukocytic packages of healthy donors, ALL UNDER ASEPTIC CONDITIONS, the cells are broken, suspensions are made adjusting volumes, and by ultracentrifugation, the suspension of cellular debris is clarified (cellular detritus), the oligopeptides are recovered by means of diafiltration and concentrated by tangential ultrafiltration. The product is formulated based on its formula of finished product in pharmaceutical presentation. However, a person skilled in the art knows that the use or utilization of human blood and its distilled derivatives or components separated from chemical procedures, cannot be commercialized, since different legislations in the world forbid it and it is classified as a crime the marketing of human blood and its derivatives. Likewise, the units of transfer factor obtained for each 450 ml of blood of healthy donors of the present invention, although is higher than the units of transfer factor obtained from egg yolk and colostrum, is only 10⁸ leukocytes×mm³.

Likewise, the document WO 97/12915, PROCEDURE FOR PURIFICATION OF THE TRANSFER FACTOR FROM LEUKOCYTES; denotes a process of purification of the Transfer Factor (oligopeptides of 1,000 to 10,000 daltons, which possess biological activity), from leukocytes, which comprises the following steps: the cells are lysed in sterile conditions, the suspension is clarified by ultrafiltration, the Transfer Factor is recovered by diafiltration and is concentrated by tangential ultrafiltration. The Transfer Factor is used pharmaceutically as a regulator of the immune response. However, this document is related with the document MX 9504215 and has the same problem of using human blood as a leukocyte extraction medium.

The patent MX20089296A, OPTIMISED PROCESS FOR THE OBTENTION OF DIALYZABLE LEUKOCYTE EXTRACT, CONTAINING PEPTIDES WITH MOLECULAR WEIGHT EQUAL TO OR LOWER THAN 10, 000 DALTONS, FROM CROCODILE LYMPHOID TISSUE AND THE PREPARATION THEREOF IN AN ORAL AND/OR INJECTABLE PHARMACEUTIC, refers to a optimized procedure for obtaining the leukocyte extract containing peptides with molecular weight equal to or less than 10,000 Daltons, from cells of lymphoid tissue from crocodile and which has the property of regulating the immune response in humans and animals. However, the power of the transfer factor obtained is 10⁸ leukocytes×mm³.

Equally, in the prior art there is the document: US 2009/0053197 TRANSFER FACTOR COMPOSITIONS AND METHODS, which mentions the possibility of using transfer factor as adjuvant, whose main feature is that the composition comprising transfer factor and an antibody, each is derived independently of a source selected from the group consisting of eggs, colostrum, blood, and combinations thereof. This means that the leucocyte extract has as source eggs, colostrum, blood or combinations thereof. However, for a person skilled in the art, it is well known that this patent presents the following problems, if egg yolk is used, it is very difficult to eliminate the lipids it contains, likewise, both the egg yolk, and the other sources consist of a low amount of leukocytes×mm³, which reduces the power to said invention. Likewise, the synthesis of the previous sources of leukocyte extract is very complicated to separate leukocyte cells from proteins, lipids, carbohydrates and toxins.

Finally, it is mentioned the documents: WO2008/042604. IMMUNE MODULATORS, PREPARATIONS AND COMPOSITIONS INCLUDING IMMUNE MODULATORS, TESTS FOR EVALUATING THE ACTIVITY OF IMMUNE MODULATORS AND PREPARATIONS AND COMPOSITIONS INCLUDING THE SAME, AND METHODS. Which discloses compositions including extracts from sources of immune modulators that include the immune molecules of the nanofraction modulator (ie, molecules that have molecular weights of about 3,000 DA and less). These compositions may also include other immune modulators, such as transfer factor. U.S. Pat. No. 4,468,379, LEUKOCYTE EXTRACTS FOR AFFECTING THE IMMUNE SYSTEM. Which describes a group of substances derived from human leukocytes (white blood cells) that amplify, suppress, or otherwise modulate the response of the immune system to the reintroduction of antigens. And U.S. Pat. No. 5,840,700, METHODS OF PRODUCING TRANSFER FACTOR. Which discloses that the invention relates to the substantially pure transfer factor with a specific activity of at least 5000 units per unit of absorption at 214 nanometers. The current invention also relates to a process for preparing the cell lysate transfer factor. The present invention includes the use of the substantially pure transfer factor with a specific activity of at least 5000 units per unit of absorption at 214 nm to treat infectious diseases.

Currently, a leukocyte source is not available for the production of potentiated TCM and without adverse effects. It has been tried to use transfer factor from egg yolk, milk and blood, wherein although the product acts as an immunomodulator, there is a low concentration of leukocytes in said sources, which translates into a low cellular excitation and deficient coupling to chemical signals. Also, these sources for extracting leukocyte extract, when administered, can cause allergic reactions to patients, such is the case of the egg. All of the above, although with different methods of synthesizing the leukocyte extracts, use as a leukocyte source either eggs of mammals, fish or birds, colostrum, blood or their combinations. However, for a person skilled in the art, it is well known that prior patents generally present the following problems, if egg yolk is used, it is very difficult to eliminate the lipids it contains, likewise, both the yolk egg, as the other sources consist of a low amount of leukocytes×mm³, which reduces the power to said invention. Likewise, the synthesis of the previous sources of leukocyte extract is very complicated to separate leukocyte cells from proteins, lipids, carbohydrates and toxins. In the same way, it is important to mention that various legislations in the world prohibit the commercialization of human blood and their derivatives. No invention was found in the prior art that refers to a method for the leukocyte counting and verification of the activated chemical signals of the leukocyte extract.

Therefore, numerous clinical trials have been carried out in the last 3 decades using extracts of white blood cells known as “transfer factor”. Unfortunately, the different scientific groups working in this field were not able to consistently generate purified and reproducible preparations of the transfer factors. Despite this, the efficacy of transfer factors have been published in revised publications for the treatment of conditions ranging from multiple sclerosis, cancer, and herpes infections. Therefore, while immunologists have always believed in the potential of transfer factors, commercial translation has been impossible due to inconsistencies in the production.

In this sense, the inventors of the present invention were able to develop an economic and scalable protocol for the generation of an optimized transfer factor from shark leukocytes. Unlike previous versions of transfer factors, TCM is characterized at molecular level and its main means of inducing therapeutic effects have been elucidated.

Clinical data have been generated in a variety of immunologically associated conditions including multiple sclerosis, viral infections and cancer. The in vitro data demonstrates the consistent production of immune modulating cytokines, including interferons and interleukins after treatment of immune cells with TCM.

Similarly, the Kirkpatrik experiments demonstrated that all the Transfer Factors described up to now are approximately 200 molecules of molecular weight less than 10,000 Kda and are a peptide of 44 amino acids, which have a common region of 10 amino acids, so that a Transfer Factor can be used from one species to another.

Another important experiment was that using Balb-c mice without any previous stimulation, were inoculated orally and at different times blood was obtained from the mice, so that with the serum in a microarray technique the interleukin excited will be searched, finding that different molecules have a specificity to excite different interleukins.

With the information up to this moment the possibility of having a specific Transfer Factor towards certain interleukins was opened and knowing that interleukin is associated with certain pathology, then we can have a Transfer Factor towards a particular pathology.

Advancing the knowledge about the interleukins network and cooperation between them, was forcing to have a Transfer Factor that covers the widest range of interleukins, so M-PLUS appeared whose formula includes the possibility of having the Transfer Factor more complete described so far.

M-PLUS was used in patients candidates to receive Cell Therapy with stem cells, shortening the response times, achieving cell differentiation in less time.

Another important achievement is the increase of NK Cells in patients with Cancer, until an increase of 30-40% is achieved and as a consequence the tumors are reduced in size, encapsulated and become operable.

Returning to the original definition of Transfer Factor which is: Dialyzable Extract of Leukocytes of a person who has a disease (example used was Tuberculosis) with positive PPD, when inoculated in a person PPD negative becomes positive PPD, concluding that the Cellular Immunity can be transferred from one person to another, but there is a premise that it is a prior induction.

The experiment performed with non-immunized mice, which resulted in a regulation of the T lymphocytes by observing the excitation of interleukins, indicates us that it refers to another function which is not that of the original Transfer Factor, that is to say, a T cell modulator or TCM, which has the function of modulating the immune response through the activation of specific molecules involved in the control of innate immunity called “Toll-like Receptors”.

T Cell Modulator (TCM) is a leukocyte extract manufactured by a proprietary methodology, described in part in the Patent Cooperation Treaty application # PCT/MX2012/000084. Therapeutic effects of TCM have previously been reported in immunologically mediated conditions, as well as oncology. Given the breadth of claimed clinical immune modulatory activities, as well as the methodology utilized in its production, we conducted a series of experiments to immunologically evaluate TCM in vitro.

Initial experiments assessed possible direct effects of TCM on cellular proliferation, given the reported antitumor activity, effects of TCM were assessed on HeLa cells, a cervical cancer cell line which has been utilized in cancer research for decades as an in vitro model of neoplasia. Originally derived in 1955, these cells are commonly utilized not for assessment of potentially useful anticancer agents, but also to assess non-specific inhibitory/cytotoxic activity of test compounds.

Production of the Th1 immunological cytokine interferon gamma (IFN-g) from human peripheral blood mononuclear cells (PBMC) was assessed directly by TCM, as well as the Th2 cytokine interleukin-4 (IL-4). In order to recapitulate in vivo effects assessment was performed of direct TCM stimulation of cytokine production, as well as addition of TCM to known cytokine inducer concanavalin A (ConA). IFNγ, is a cytokine that is critical for innate and adaptive immunity against viral and intracellular bacterial infections and for tumor control. IFNγ is an important activator of macrophages. Abnormal IFNγ expression is associated with a number of autoinflammatory and autoimmune diseases. The importance of IFNγ in the immune system stems in part from its ability to inhibit viral replication directly, and most importantly from its ability to stimulate and/or regulate the immune system. IFNγ is produced predominantly by natural killer (NK) and natural killer T (NKT) cells as part of the innate immune response, and by cytotoxic T lymphocyte (CTL) effector T cells. IL-4 is considered a prototypic Th2 cytokine, important in stimulation of antibody mediated immune responses, as well as generation of plasma cells. IL-4 is important in stimulating anti-inflammatory responses and has been used successfully in treatment of the mouse model of Type 1 diabetes, as well as other anti-inflammatory diseases. Accordingly, we assessed production of these two cytokines to gather an idea whether TCM acts on Th1 or Th2 cells, which is the broad classification of immune responses.

T cell activation, and subsequent polarization into Th1 or Th2 subsets is controlled by mature dendritic cells which provide costimulatory molecules, in addition to antigen-MHC signals to the T cell. We sought to determine whether TCM induced maturation of dendritic cells, and assessed one of the major molecules involved in dendritic cell maturation, Toll like receptor (TLR)-4.

Given the reported dual roles of TCM in both immune stimulation (eg anticancer and antiviral) as well as immune regulation (efficacy in vitiligo and dry eye), and could be important for HIV patients and different cancer treatment by Dendritic Cells, we sought to determine whether TCM affected the generation of T regulatory cells.

Therefore, the TCM is a low molecular weight leukocyte lysate-based immune modulator produced under GMP and currently used in a variety of veterinary and clinical settings as a nutraceutical. Although numerous experiments have been performed during its cellular and immunological properties of commercially-available TCM. We demonstrated that TCM did not exhibit cytotoxic or antiproliferative effects even at supraphysiological concentrations in both cancerous and non-cancerous cell lines. Evaluation of cytokine modulatory activity of TCM revealed a potent costimulatory activity on interferon gamma (IFN-g) and interleukin-4 (IL-4) production from peripheral blood mononuclear cells (PMBC) stimulated with concanavalin A (Con A) or phytohemagglutinin (PHA). Given that PBMC possess antigen presenting cells that may be acting as costimulators, we assessed effects of TCM on stimulation of dendritic cell (DC) maturation. TCM stimulated expression of DC maturation markers CD80 and CD86, as well as upregulated production of both IL-10 and IL-12: key cytokines controlling T cell activity. Blockade of TLR-4 using LPS-RS resulted in inhibition of TCM-induced changes in DC maturation. These data demonstrate TCM possesses potent immune modulatory activity at the level of antigen commercial development, these have been kept as trade secrets. This has resulted in a lack of mechanistic knowledge regarding TCM in the peer-reviewed literature. The purpose of this study is to characterize presentation, which is a key component of immune surveillance.

### OBJECTS OF THE INVENTION

Therefore, it is an object of the present invention to provide a source of leukocyte extract whose origin is not from mammals, eggs, nor colostrum, and which provides a potency of 10¹² leukocytes×mm³ (understanding by potency to the amount of leukocytes and quality of smooth, round and innocuous cells), necessary amount for cellular excitation and optimization of chemical signals.

A further object of the present invention is to obtain a TCM and its use for the consistent production of immune modulating cytokines, including interferons and interleukins.

Another additional object of the present invention is to obtain a TCM for use as a promoter of the increase and activity of NK (Natural Killer) cells which provide protection against viruses as part of the natural immune defense system,

Still another additional object of the present invention is the obtaining of a TCM for increasing the antigen presentation and the lysosome activity of the macrophages (allowing the destruction of foreign bodies), promoting the adhesion and the necessary binding for the migration of leukocytes (allows the immune cells to travel more freely) and as an inducer of antiviral effects.

Another object of the present invention is to obtain a TCM and its pharmaceutical use for the treatment of cervical cancer, in particular the cell line of HeLa cells.

It is also an object of the present invention to provide an increase in NK cells in patients with cancer, until achieve an increase of 30-40% and consequently to reduce tumors size, so that they encapsulate and become operable.

Therefore, the present invention refers to a leukocyte extract containing polypeptides equal to or less than 10,000 daltons from cells, tissues or organs of selacimorphs, in specific but not limiting shark spleen and its use as cellular exciter and enhancer of chemical signal in the body, that is, optimizing the natural immune system of the individual.

Likewise, it is also an object of the present invention to provide a method for the counting of leukocytes and verification of the activated chemical signals of the leukocyte extract.

The additional aspects of the invention relate to compositions and formulations which may comprise components in powder, encapsulated presentation, including, but not limited to, T cell modulator in several presentations, either encapsulated or powdered for its pharmaceutical use in the treatment of cervical cancer in particular the HeLa cell line.

### BRIEF DESCRIPTION OF THE FIGURES

FIG. 1 shows a comparison between the concentrations of the transfer factor of the extraction means of the prior art and the TCM of the present invention.

FIG. 2 shows the method of checking the power of the leukocyte extract from the inoculation of the leukocyte extract in Balb-c mice.

FIG. 3 shows how the TCM does not affect the HeLa Cancer Cell Proliferation in Experiment 1 (AXIS X vs AXIS Y).

FIG. 4 shows how the TCM does not affect the proliferation of HeLa Cancer Cells in Experiment 2 (AXIS X vs AXIS Y).

FIG. 5 shows how the TCM does not affect the HeLa Cancer Cell Proliferation in Experiment 3 (X AXIS vs AXIS Y).

FIG. 6 shows how TCM increases the production of ConA induced by interferon in Human PBMC in Experiment 1 (AXIS X vs AXIS Y).

FIG. 7 shows how the TCM increases the production of ConA induced by interferon in Human PBMC in Experiment 2 (AXIS X vs AXIS Y).

FIG. 8 shows how TCM increases the production of ConA induced by interferon in Human PBMC in Experiment 3 (AXIS X vs AXIS Y).

### DETAILED DESCRIPTION OF THE INVENTION

The method of the present invention for the extraction of a dialyzable extract of leukocytes is the following:

1. Sterilization.—Involves that any instrument used to extract the leukocyte extract should be sterile.

2. Extraction of spleen. This step consists of extracting the shark spleen surgically in order to extract the leukocyte extract.

3. Counting and quantification.—By means of the Neubauer chamber and microscope, the number of leukocytes per field in the Neubauer grid is counted, to know the power of the T-cell modulator that will be obtained, that is, the number of leukocytes per cubic millimeter, adjusting the counting to 10¹² leukocytes×mm³, by adding shark spleen to achieve the count of 10¹² leukocytes×mm³. Likewise, it is necessary to evaluate the quality of said cells, so that there is no anisocytosis, that is, through the microscope it is observed that the cells are round and smooth.

4. Breaking or separation of components.—The leukocyte cells must be separated from proteins, lipids, carbohydrates and toxins.

5. Dialysis.—Dialysis is the process of separating the molecules in a solution by the difference in their diffusion rates through a semipermeable membrane. Then, the leukocyte extract, after the separation and breaking of components has been performed, is placed in a semipermeable dialysis bag, for example, in a membrane of the cellulose with pores, and the bag is sealed. The sealed dialysis bag is placed in a vessel with a different solution, or pure water. Due to the fact that the leukocyte extracts, is small enough to pass through the pores tend to move inwards or outwards of the dialysis bag in the direction of the lowest concentration. The larger molecules (often proteins, DNA, or polysaccharides) which have dimensions significantly larger than the pore diameter are retained within the dialysis bag. In this way, leukocyte extracts less than or equal to 10,000 Daltons are separated.

6. Filtration and sterilization.—After the dialysis, the leukocyte extract is filtered by means of a membrane of pore size between 2 and 4 micrometers. Likewise, the solution is sterilized again.

7. Formulation.—A lyophilization process is carried out to remove the water from the leucocyte extract by means of the generation of a vacuum, likewise in this step the aggregation of a vehicle is carried out, such as milk, water, gel or artificial flavoring, to give a presentation and pleasant taste to the product.

8. Physical-chemical evaluation. At this point the physical-chemical processes such as density, pH, color, smell and taste are evaluated. It is important to mention that if no vehicle was added to the product, the T-cell modulator obtained will be odorless, colorless and tasteless.

9. Evaluation of biological activity. The leukocyte extract is analyzed by inoculating the extract in Balb-c mice at a concentration equivalent to that used in humans in relation to weight-leukocyte extract, an inoculation kinetic is performed by having mice exposed to the extract during a determined period of time, for example 0, 2, 6, 24, 48 and 120 hours; blood is extracted from the mouse, from which the serum will serve for the determination of activated cytokines placing serum on microarray membranes to determine the type of cytokine found in the leukocyte extract and the time of activity of the chemical signal in the induction of cytokines. Also making dilutions of the serum until find the point where the cytosine is no longer found, which means that the last dilution is the title of the present cytosine. This means that the greater the title or dilution factor, the greater the power of the leukocyte extract. By means of the study in times, that is to say the kinetics, it will be indicative of the start time of the induction of cytokines, the optimal time of induction of cytokines and the time of total permanence of the induction of cytokines.

The result of the previous process is a potentiated T-cell modulator in powder form, which provides it the virtue of being easily transported and stored, it does not require refrigeration and a power of T cell modulator of 10¹² leukocytes×mm³ is obtained, which is highly superior to any known T-cell modulator, being understood by potency the concentration of leukocytes per mm³ and the quality of the cells (smooth, round and innocuous).

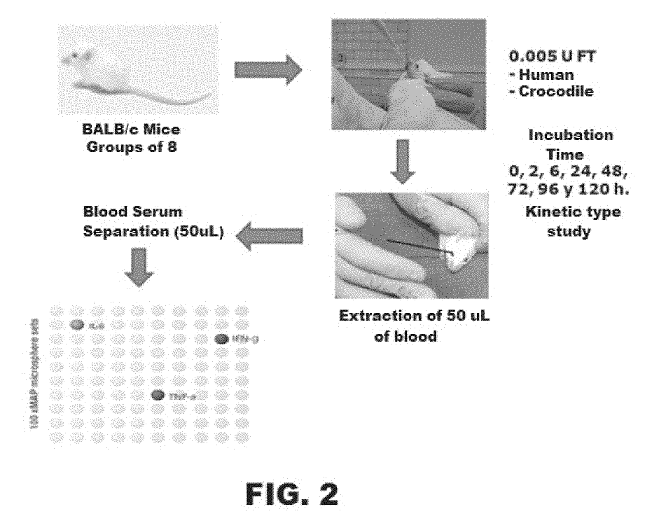

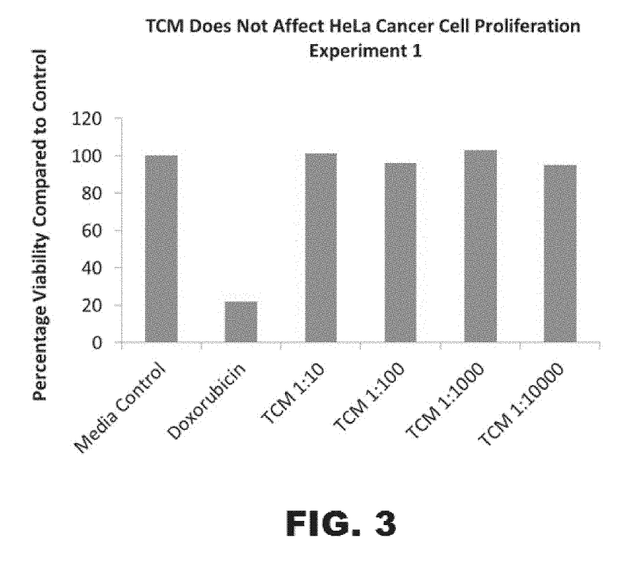

The present FIG. 2 illustrates the method of checking the power of the leukocyte extract from the inoculation of the leukocyte extract in Balb-c mice. Groups of 8 mice are used, which will be used in each time of the kinetics, will be inoculated with the amount equivalent to the weight-unit relationship of T-cell modulator (0.005 unit of T-cell modulator), also using cell modulator T from human leukocyte and from crocodile spleen for comparative purposes, the mice are maintained at the determined times, being time 0 the basal level of induced cytokines of the mice, which will be eliminated with the intention of knowing the type, Title and permanence of the induced cytokines.

Serum is extracted from each mouse in its time according to the kinetics and 50 microliters of serum are used, they are exposed to the microarray membranes containing the receptor antibodies of the cytokines and the wells that develop color will be the induced cytokines. The serum is diluted with a regulating solution in multiples of 2 initially so as to subsequently carry out dilutions in multiples of 100. The basal dilution of time 0 is eliminated and the dilution that preserves the color development in the microarrays prior to dilution where it is no longer present the color development, is the title of the leukocyte extract. The group of mice that retains the maximum title with the longest induction time will be the residence time of the cytosine induction.

Thus, with the experiment using Balb-c mice without any previous stimulus, we searched for the interleukin excited, finding that different molecules have a specificity to excite different interleukins.

Based on the foregoing, it was determined that the potentiated TCM of the present invention has the function of:

Promoting the activity of the NK (natural killer) cells, which provide protection against viruses as part of the natural immune defense system.

Producing consistently immune modulating cytokines, including interferons and interleukins.

Increasing antigen presentation and lysosome activity of macrophages (allowing the destruction of foreign bodies).

Promoting the adhesion and union necessary for the migration of leukocytes (allows the immune cells to travel more freely) and as inducer of antiviral effects.

Likewise, in another aspect of the present invention the TCM is designed for pharmaceutical use in the treatment of cervical cancer in particular the cell line of HeLa cells, which are commonly used not only for the evaluation of potentially useful anticancer agents, but also to evaluate the non-specific inhibitory/cytotoxic activity of the test compounds.

In our study, the alteration of HeLa cell proliferation was evaluated on screen for the possible direct anti-cancer/cell inhibitory activity of the TCM. In this sense, if inhibitory effects are present, the next step of the experiment would be to evaluate if the inhibition is directly towards the tumor cells, or if it is also against non-tumor control tissue.

PBMC Interferon Gamma

Interferon gamma (IFNγ) is a cytokine (broad and loose category of small proteins that are important in cell signaling). It was first recognized when human or mouse lymphocytes obtained from TB positive individuals were exposed to a PPD skin test. This interferon was later called macrophage-activating factor, a term now used to describe a larger family of proteins to which IFNγ belongs. In humans, the IFNγ protein is encoded by the IFNG gene.

IFNγ, is a cytokine that is critical for innate and adaptive immunity against viral and intracellular bacterial infections and for tumor control. IFNγ is an important activator of macrophages. Abnormal IFNγ expression is associated with a number of autoinflammatory and autoimmune diseases. The importance of IFNγ in the immune system stems in part from its ability to inhibit viral replication directly, and most importantly from its ability to stimulate and/or regulate the immune system. IFNγ is produced predominantly by natural killer (NK) and natural killer T (NKT) cells as part of the innate immune response, and by cytotoxic T lymphocyte (CTL) effector T cells.

IFNγ has antiviral, immunoregulatory, and anti-tumor properties. It alters transcription in up to 30 genes producing a variety of physiological and cellular responses. Among the effects are:
Promotes NK (natural killer) cells activity
Increase antigen presentation and lysosome activity of macrophages.
Promotes adhesion and binding required for leukocyte migration (allows immune cells to travel more freely)
Induces antiviral effects.

For a better understanding of the invention, illustrative examples of the use and application thereof are shown below.

### Example 1

Product 2 times concentrated (0.520 g) lanes: 1.—Molecular weight marker 2.—negative control 3.4.5.—product twice concentrated.

The product weighs less than 10,000 KDa.

To study the effect of Vitikare in animal models and at the same time demonstrate the effect of induction of cytokines, the following experiment was carried out:

Balb/c mice were selected in groups of 8, from which a group was left as control and to 8 groups of 8 mice each were administered 0.005 U of Transfer Factor of human origin and blood was extracted via ocular at 0, 2, 6, 24, 48, 72, 96 and 120 hours to be examined by microarrays, the ability to induce cytokines.

In another 8 groups of 8 mice each was supplied with a TCM of shark spleen origin following the same procedure as the Human Transfer Factor. It was found that the Human Transfer Factor induces the production of cytokines IL-2, IFNγ and TNFα as indicated in the literature, but the shark TCM induces the same cytokines, also annexing the cytokines indicated in FIG. 2.

For the determination of TCM potency, mouse serum dilutions are made (multiples of 1:100) until the disappearance of the cytokine induction, being the last dilution up to where the cytokine induction appears as the power title of the TCM.

List of Induced Cytokines in this experiment can be seen in the following Table.

| CYTOKINE | CYTOKINE |
|---|---|
| EOTAXIN | IL-15 |
| G-CSF | IL-17 |
| GM-CSF | IP-10 |
| INF-γ | MIP-2 |
| IL-1α | KC |
| M-CSF | LIF |
| IL-1β | LIX |
| IL-2 | MCP-1 |
| IL-3 | MIP-1α |
| IL-4 | MIP-1β |
| IL-5 | MIG |
| IL-6 | RANTES |
| IL-7 | TNFα |
| IL-10 | IL-12 (P70) |
| IL-12 (p40) | VEGF |
| IL-13 | IL-9 |

### Example 2

Protocol

Once obtained a TCM sample, the next step is to obtain HeLa human cervical cancer cells from American Type Tissue Culture (ATCC: Manassas, Va.) and grown under fully humidified 5% CO2 environment with MEM supplemented with 10% FBS, 2% sodium pyruvate, non-essential amino acids (2 mM), penicillin (100 units/ml), streptomycin (100 μg/ml), and glutamine (4 mM) (Gibco-BRL). Cells were passaged by trypsinization twice weekly or as needed based on 75% confluency

Likewise, a sample of peripheral blood mononuclear cells (PBMC) was obtained, which was isolated from buffy coats by density-gradient centrifugation. Specifically, buffy coat cells were dispensed over five 50 ml falcon tubes, phosphate-buffered saline (PBS)/2% fetal calf serum (FCS) solution was added to reach a volume of 20 ml and 10 ml Ficoll-Paque® was gently added under the diluted buffy coat cells. Centrifugation was performed at 400 g for 20 min at room temperature (RT) and washing of PBMC was done three times with PBS/2% FCS. Culture of freshly isolated PBMC was performed in complete MEM media.

TCM was diluted in complete MEM media prepared as described above. Dilutions of 1:10, 1:100, 1;1000 and 1:10,000 were performed. Negative controls were complete MEM media. Positive controls were concanavalin A at a concentration of 2.5 ug/ml. PBMC were plated at 1.5×10⁶ cells/ml in flat-bottom 96-well culture plates in a volume of 200 μl per well and incubated at 37° in a humidified 5% CO₂ atmosphere. Conditioned media was then evaluated for IFN-gamma production using ELISA from R & D Systems (Quantikine ELISA). Concentration was calculated by plotting against a standard curve generated with control cytokine.

HeLa cells were plated at a concentration of 10,000 cells per well in flat bottom plates and incubated with dilutions of TCM at 1:10, 1:100, 1;1000 and 1:10,000. The 3-(4,5-dimethylthiazol-2-yl)-2,5-diphenyl tetrazolium bromide (MTT) assay was performed for assessment of proliferation. In this assay soluble MTT is metabolized by mitochondrial enzyme activity of viable tumor cells, into an insoluble colored formazan product. Subsequently formazan were dissolved in DMSO and measured spectrophotometrically at 540 nm. Briefly, 200 μl of cell suspension was seeded in 96-well microplates and incubated for 48 h (37° C., 5% CO₂ air humidified).

To evaluate cell survival, 20 μl of MTT solution (5 mg/ml in PBS) was added to each well and incubated for 3 h. Then gently 150 μl of old medium containing MTT was replaced by DMSO and pipetted to dissolve any formed formazan crystals. Absorbance was then determined at 540 nm by enzyme-linked immunosorbent assay (ELISA) plate reader. Each extract concentration was assayed in 4 wells and repeated 3-times.

1. TCM does not affect viability of proliferation of HeLa cancer cells.

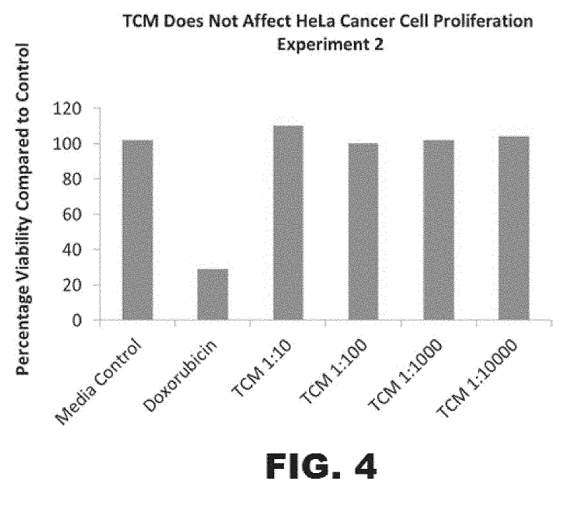

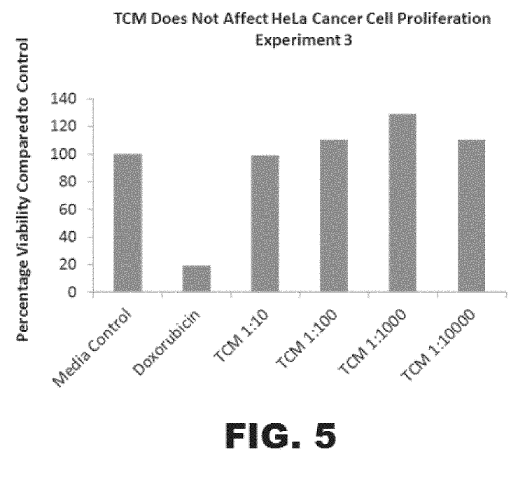

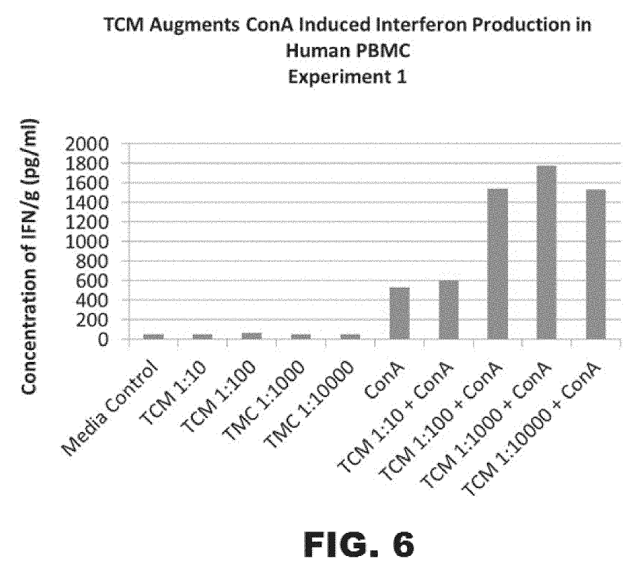

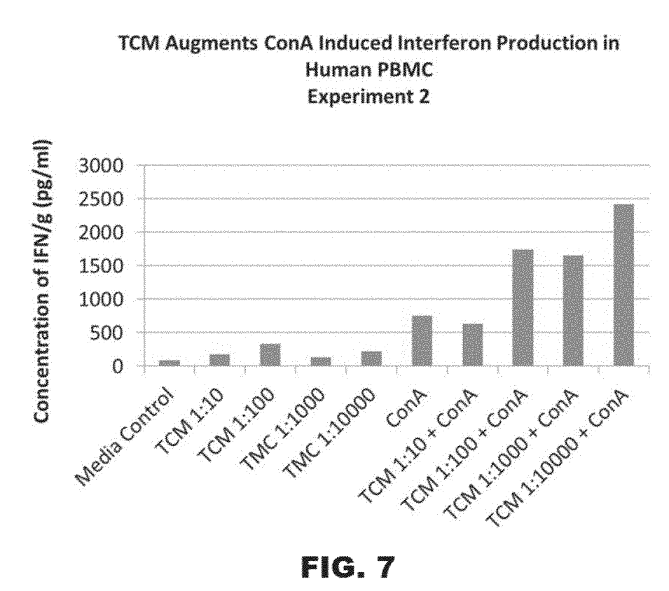

With reference to FIGS. 3 to 5, it does not appear that TCM has any antimitotic effects on the cancer cell line HeLa. This is consistent with the notion of TCM being an immune stimulator, in contrast to potential for being a direct cancer toxic drug.

2. TCM Stimulates Interferon Production from Activated T cells

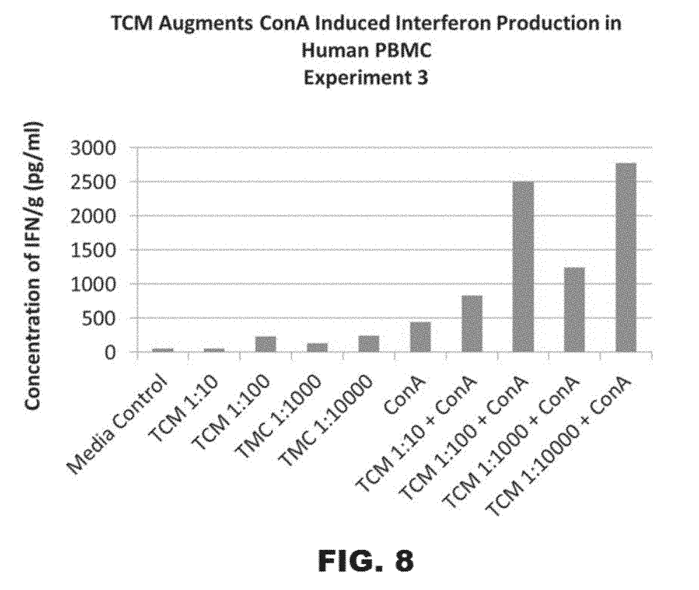

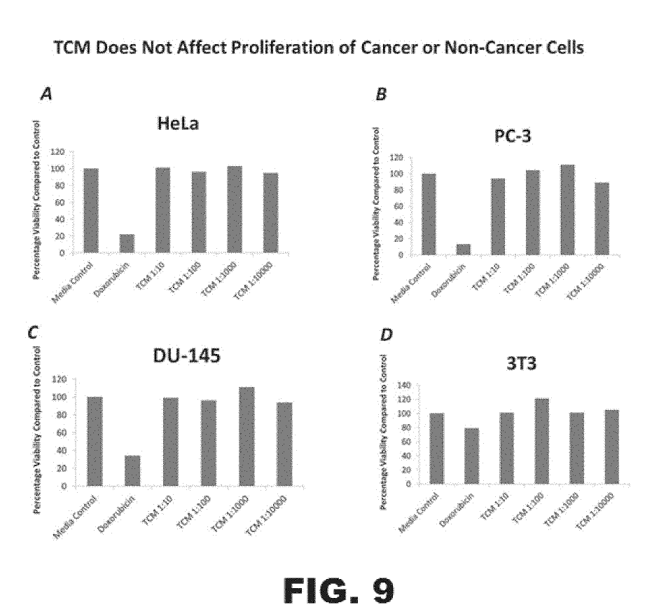

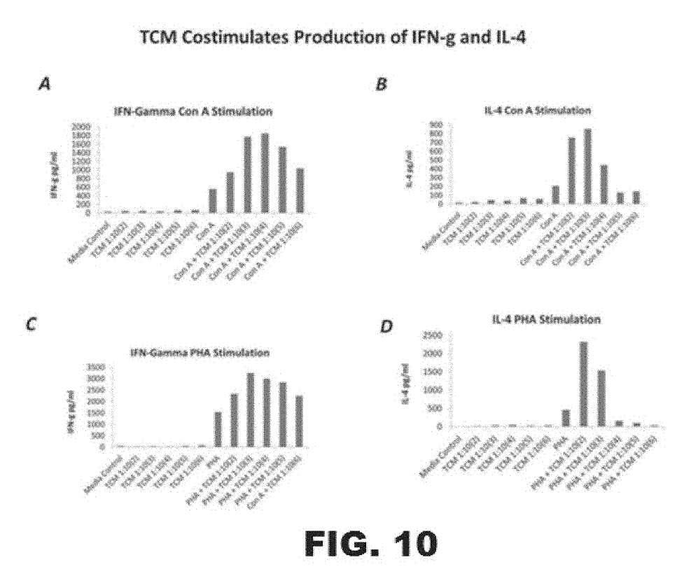

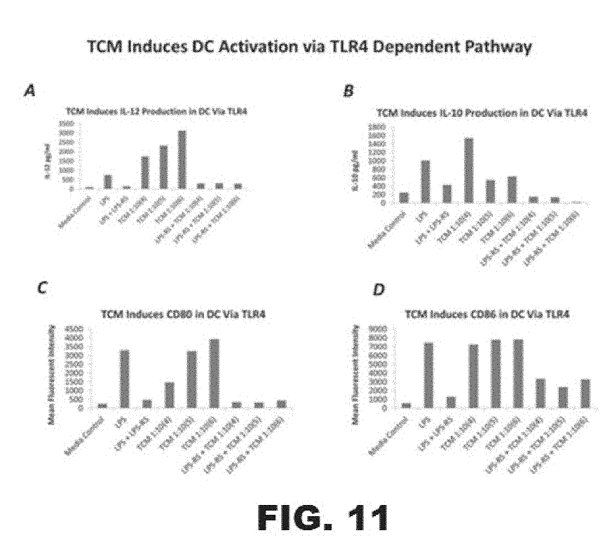

To assess whether TCM directly activates T cell production of cytokines, or whether it requires a costimulatory signal, such as ConA, was examined. With reference to FIGS. 6 to 8, it appears that TCM adds to existing immunity, but does not initiate immunity, at least based on IFN-gamma assays. This would be consistent with an immune regulatory role of IFN-gamma.

Conclusions

Therefore, the demonstration of a non-cytotoxic nature of TCM, even at high concentrations (1:10 dilution) suggests that relative innocuous nature of the composition. Additionally, it suggests that biological activities are mediated by immune modulation, as oppose to direct effects on cell mitogenesis.

Augmentation of IFN-gamma synthesis in response to ConA, supports the notion that TCM is an immune modulator, providing support to immune responses that have already initiated. This is an important characteristic since direct stimulation of interferon gamma, in absence of other stimulation would implicate the possibility that TCM would induce toxicity that has been associated with other immune modulators such as IL-2 administration.

The current data provide a scientific mechanism for immune modulatory activities of TCM, and support its use as a “support mechanism” to the immune response, said data can be appreciated in FIGS. 2 to 7.

### Example 3

TCM TCM is extracted from Lysed Shark Spleen Leukocytes utilizing dialyzable process. TCM was obtained from a manufacturer.

Cell Lines

HeLa human cervical cancer cells were obtained from American Type Tissue Culture (ATCC: Manassas, Va.) and grown under fully humidified 5% CO2 environment with MEM supplemented with 10% FBS, 2% sodium pyruvate, non-essential amino acids (2 mM), penicillin (100 units/imp, streptomycin (100 μg/ml), and glutamine (4 mM) (Gibco-BRL). Cells were passaged by trypsinization twice weekly or as needed based on 75% confluency

Peripheral Blood Mononuclear Cells (PBMC)

PBMC were isolated from buffy coats by density-gradient centrifugation. Specifically, buffy coat cells were dispensed over five 50 ml falcon tubes, phosphate-buffered saline (PBS)/2% fetal calf serum (FCS) solution was added to reach a volume of 20 ml and 10 ml Ficoll-Paque® was gently added under the diluted buffy coat cells. Centrifugation was performed at 400 g for 20 min at room temperature (RT) and washing of PBMC was done three times with PBS/2% FCS. Culture of freshly isolated PBMC was performed in complete MEM media.

Cell Treatments and Analysis

TCM was diluted in complete MEM media prepared as described above. Dilutions of 1:10, 1:100, 1;1000 and 1:10,000 were performed. Negative controls were complete MEM media. Positive controls were concanavalin A at a concentration of 2.5 ug/ml. PBMC were plated at 1.5×106 cells/ml in flat-bottom 96-well culture plates in a volume of 200 μl per well and incubated at 37° in a humidified 5% CO2 atmosphere. Conditioned media was then evaluated for IFN-gamma production using ELISA from R & D Systems (Quantikine ELISA). Concentration was calculated by plotting against a standard curve generated with control cytokine.

HeLa cells were plated at a concentration of 10,000 cells per well in flat bottom plates and incubated with dilutions of TCM at 1:10, 1:100, 1;1000 and 1:10,000. The 3-(4,5-dimethylthiazol-2-yl)-2,5-diphenyl tetrazolium bromide (MTT) assay was performed for assessment of proliferation. In this assay soluble MTT is metabolized by mitochondrial enzyme activity of viable tumor cells, into an insoluble colored formazan product. Subsequently formazan were dissolved in DMSO and measured spectrophotometrically at 540 nm. Briefly, 200 μl of cell suspension was seeded in 96-well microplates and incubated for 48 h (37° C., 5% CO2 air humidified).

To evaluate cell survival, 20 μl of MTT solution (5 mg/ml in PBS) was added to each well and incubated for 3 h. Then gently 150 μl of old medium containing MTT was replaced by DMSO and pipetted to dissolve any formed formazan crystals. Absorbance was then determined at 540 nm by enzyme-linked immunosorbent assay (ELISA) plate reader. Each extract concentration was assayed in 4 wells and repeated 3-times.

ELISA

IFN-gamma, IL-4, IL-10 and IL-12 were assessed by ELISA (R and D Systems) utilizing supernatant from mitogen activated cultures and treated DC.

Dendritic Cells

DC were generated from PBMC resuspended in RPMI-10% FCS, and allowed to adhere to 6-well plates (Costar Corp., Cambridge, Mass.). After 2 h incubation at 37 Celsius, the nonadherent cells were removed and the adherent cells washed in phosphate buffered saline (PBS), followed by detachment by incubation with Mg 2+ and Ca 2+ free PBS containing 0.5 mM EDTA at 37 Celsius. The adherent fraction was subsequently cultured at 3×10(6)/ml in RPMI-10% FCS supplemented with 50 ng/ml GM-CSF and 1,000 U/ml IL-4. Media is changed every 2 days for a total of 8 days culture. DC were isolated by positive selection for CD83 and subsequently treated with TCM on day 6 of culture. Assessment of maturation was performed by flow cytometry for CD80 and CD86 expression.

Blockade of TLR-4 was performed using by culture in the presence of TLR4 antagonist LPS-RS (Invivogen (San Diego, Calif.), (5 μg/mL), with pretreatment 4 hours before exposure to TCM.

According to the previous, the following results were obtained:

1. TCM does not Modulate Cellular Proliferation

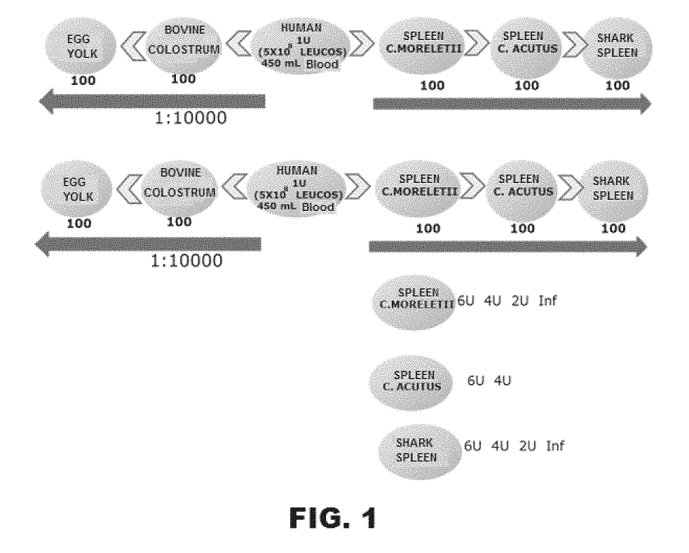

TCM has been reported to possess anticancer activity. Accordingly, we conducted a series of experiments assessing ability of various concentrations of TCM to inhibit proliferation of HeLa cells. We utilized the chemotherapeutic drug doxorubicin as a control. As seen in FIG. 1a, various doses of TCM did not affect proliferation of HeLa cells as assessed in the MTT assay after 48 hours of culture. Importantly, supraphysiological doses of TCM, as high as 1:10 diluted volume by volume in the tissue culture media did not result in inhibition of proliferation. These data suggest that TCM does not act through cytotoxic or cytostatic mechanisms. These data were confirmed with other cell lines such as PC-3, DU-145, and non-malignant 3T3 fibroblasts (FIGS. 9b-9d).

2. TCM Acts as a Cofactor for Cytokine Secretion from Immune Cells Found in Peripheral Blood

To assess whether TCM directly activates T cell production of cytokines, or whether it requires a costimulatory signal, such as concanavalin A (ConA), was examined. TCM did not affect viability of PBMC (data not shown). It appears that TCM complements existing production of immune stimulatory molecules after a primary stimuli, but does not initiate immunity, at least based on IFN-gamma and IL-4 production (FIGS. 10a and 10b). Given that different doses of TCM possess different costimulatory profiles for the different cytokines, we questioned whether the effect was specific to conconavalin A stimulation, or whether other factors may be at play. Accordingly, we substitute stimulation by conconavalin A to stimulation by phytohemagglutinin, a mitogen often used in studies stimulating human T cells. As seen in FIGS. 2c and 2d, a similar pattern of IFN-gamma and IL-4 costimulation was observed with PHA acting as the primary stimulator.

3. TCM Induces Dendritic Cell Maturation in a TLR4 Dependent Manner

Given the contamination of antigen presenting cells in PBMC, and the fact that antigen presenting cells may be sending costimulatory signals to the T cells in response to TCM treatment, a series of experiments were conducted to assess whether TCM acts on the most potent antigen presenting cell, the dendritic cell. Day 6 immature DC generated from monocytes by IL4 and GM-CSF treatment were used to assess maturation-inducing potential of TCM. Cells were treated with saline, Ips positive control, and 3 concentrations of TCM. Additionally, blockade of TLR4 signalling was performed by cotreatment with LPS-RS, an antagonist of the TLR-4 receptor. As seen in FIGS. 3a and 3b, TCM was capable of upregulating expression of IL-12 and IL-10, respectively, suggesting from a functional perspective that DC activation was occurring. Indeed the fact that IL-12 drives Th1 cytokine production and IL-10 drives Th2, these data are in agreement with the previous data suggesting that TCM is capable of modulating immunity. Definative evidence of maturation of DC was observed using flow cytometry, demonstrating that upregulation of CD80 and CD86 was occurring as a result of TCM treatment (FIGS. 11c and 11d). In all experiments, blockade of TLR-4 by treatment with LPS-RS, an antagonist of TLR4, resulted in marked reduction of both LPS induced changes (positive control) as well as in activity of TCM.

### CONCLUSION

TCM is a low molecular weight leukocyte extract that induces a TLR-4 dependent modulation of dendritic cell activity, which we have shown is associated with T cell cytokine production in a costimulatory manner. The ability of TCM to modulate the immune system at the level of antigen presentation supports its current use as an immunological adjuvant for vaccination, as well as its immune modulatory activity, which is observed in conditions ranging from vitiligo to viral and oncological states.

1. Potentiated T cell modulator (TCM) capable of modulating the immune response through the activation of specific molecules involved in the control of innate immunity obtained by a dialyzable extract of leukocytes from leukocyte cells containing polypeptides equal or less to 10,000 daltons, wherein the unit of transfer factor of said TCM is defined as the equivalent of 1012 leukocytes×mm3 whose specific source is the spleen of selacimorphs or sharks which is the center of activity of the immune system thereof.

2. Method for extracting, checking and counting leukocyte dialyzable extract from leukocyte cells containing polypeptides equal to or less than 10,000 daltons for obtaining a potentiated T cell modulator (TCM) able to modulate the immune response through the activation of specific molecules involved in the control of innate immunity, wherein the specific source is the spleen of selacimorphs or sharks which is the center of activity of the immune system of said selacimorphs or sharks, said method comprising the following steps:
a) sterilization.—Involves that any instrument used to extract the leukocyte extract should be sterile;
b) extraction of spleen. This step consists of extracting the shark spleen surgically in order to extract the leukocyte extract;
i. unfreezing the organ to work;
ii. disintegrating in a sterile mortar with the help of the liquid that may or may not accompany the organ, in case of not presenting it, sterile distilled water may be used; counting the number of spleen cells, adjusting the number of cells to 5×108 cells per ml, the maximum volume to be obtained is 250 ml;
iii. the liquid is distributed in new 50 ml Falcon tubes, which are centrifuged at 1500 rpm;
c) counting and quantification.—By means of the Neubauer chamber and microscope, the number of leukocytes per field in the Neubauer grid is counted, to know the power of the T-cell modulator that will be obtained, that is, the number of leukocytes per cubic millimeter, adjusting the counting to 1012 leukocytes×mm3, by adding shark spleen to achieve the count of 1012 leukocytes×mm3. Likewise, it is necessary to evaluate the quality of said cells, so that there is no anisocytosis, that is, through the microscope it is observed that the cells are round and smooth;
d) breaking or separation of components.—The leukocyte cells must be separated from proteins, lipids, carbohydrates and toxins;
e) dialysis.—separating the molecules in a solution by the difference in their diffusion rates through a semipermeable membrane. Then, the leukocyte extract, after the separation and breaking of components has been performed, is placed in a semipermeable dialysis bag, for example, in a membrane of the cellulose with pores, and the bag is sealed. The sealed dialysis bag is placed in a vessel with a different solution, or pure water. Due to the fact that the leukocyte extracts, is small enough to pass through the pores tend to move inwards or outwards of the dialysis bag in the direction of the lowest concentration. The larger molecules (often proteins, DNA, or polysaccharides) which have dimensions significantly larger than the pore diameter are retained within the dialysis bag. In this way, leukocyte extracts less than or equal to 10,000 Daltons are separated;
i. cutting a section of the membrane for dialysis previously treated keeping the following relationship: for every 5 ml of solution, 25 cm of membrane should be cut;
ii. closing the membrane with sterile hemp thread at one end (2 folds and one more on itself), every two turns a strong knot;
iii. using the same hydration fluid of the membrane, placing within the same an approximate volume of 30 ml, in order to verify that there are no leaks. At the end, the fluid is removed and filling with the supernatant from the Falcon tubes is started; Note: Avoid dehydration of the membrane since it prevents the correct distribution of the liquid;
iv. filling up to 5 or 10 cm before the end of the membrane, in which a knot similar to the other end is made; Note: Avoid the formation of bubbles, since these would hinder the dialysis process;
v. placing in a 4 liter flask an approximate volume of 2 liters of distilled water previously sterilized and filtered; before introducing into the membrane, it is recommended to rinse this with distilled water to eliminate the likely contaminants present;
vi. immediately introducing the membrane and immerse it in the distilled water, leaving attached to one end a piece of thread for its optimal handling during the dialysate; Note: the previous step should be repeated 3 times, always in a flask with different sterile distilled water;
f) filtration and sterilization.—after the dialysis, the leukocyte extract is filtered by means of a membrane of pore size between 2 and 4 micrometers. Likewise, the solution is sterilized again;
g) formulation.—a lyophilization process is carried out to remove the water from the leucocyte extract by means of the generation of a vacuum, likewise in this step the aggregation of a vehicle is carried out, such as milk, water, gel or artificial flavoring, to give a presentation and pleasant taste to the product;
h) physical-chemical evaluation. At this point the physical-chemical processes such as density, pH, color, smell and taste are evaluated. It is important to mention that if no vehicle was added to the product, the T-cell modulator obtained will be odorless, colorless and tasteless.
i) evaluation of biological activity. The leukocyte extract is analyzed by inoculating the extract in Balb-c mice at a concentration equivalent to that used in humans in relation to weight-leukocyte extract, an inoculation kinetic is performed by having mice exposed to the extract during a determined period of time, for example 0, 2, 6, 24, 48 and 120 hours; blood is extracted from the mouse, from which the serum will serve for the determination of activated cytokines placing serum on microarray membranes to determine the type of cytokine found in the leukocyte extract and the time of activity of the chemical signal in the induction of cytokines. Also making dilutions of the serum until find the point where the cytosine is no longer found, which means that the last dilution is the title of the present cytosine. This means that the greater the title or dilution factor, the greater the power of the leukocyte extract. By means of the study in times, that is to say the kinetics, it will be indicative of the start time of the induction of cytokines, the optimal time of induction of cytokines and the time of total permanence of the induction of cytokines.

3. The potentiated T cell modulator (TCM) according to claim 1, wherein said TCM is in a powder presentation, to be easily transported and stored, without requiring refrigeration.

4. The potentiated T cell modulator (TCM) according to claim 1, wherein the transfer factor equivalent to 1012 leukocytes×mm3 refers to the power in the leukocyte concentration per mm3 and the quality of the cells (smooth, round and innocuous) whose quantity is necessary for cellular excitation and optimization of chemical signals.

5. Method of checking the power of the leukocyte extract from the inoculation of the leukocyte extract in Balb-c mice, carried out in the step of checking the biological activity, which comprises: using groups of 8 mice, which will be used at each time of the kinetics, they will be inoculated with the amount equivalent to the weight-unit ratio of T-cell modulator (0.005 unit of T-cell modulator); maintaining the mice at the determined times, being time 0 the basal level of induced cytokines of the mice, which will be eliminated with the intention of knowing the type, title and permanence of the induced cytokines; extracting serum from each mouse in its time according to the kinetics and using 50 microliters of serum, exposing in front of the membranes of microarrays containing the cytosine receptor antibodies and the wells that develop color will be the induced cytokines; diluting the serum with a buffer in multiples of 2 initially and then make dilutions in multiples of 100; eliminating the basal dilution of time 0, wherein the dilution that preserves the color development in the microarrays prior to dilution where color development is no longer present, is the title of the leukocyte extract; and wherein the group of mice that retains the maximum titer with the longest induction time will be the residence time of the cytosine induction.

6. The method according to claim 5, wherein the higher the titer and time of induction found, the greater the power of the T cell modulator (TCM).

7. The method according to claim 6, wherein said TCM promotes cellular excitation and optimization of chemical signals within the organism of the individual who consumed it.

8. The potentiated T cell modulator (TCM) according to claim 1, wherein said TCM promotes the increase and activity of NK cells (Natural Killer) which provide protection against viruses as part of the immune defense system natural.

9. The potentiated T cell modulator (TCM) according to claim 1, wherein the increase of NK cells is achieved up to an increase of 30-40% resulting in the reduction of tumor size, so that they are encapsulated and become operable.

10. The potentiated T cell modulator (TCM) according to claim 1, wherein said TCM promotes the increase in the presentation of the antigen and the lysosome activity of the macrophages allowing the destruction of foreign bodies.

11. The potentiated T cell modulator (TCM) according to claim 1, wherein said TCM promotes the adhesion and necessary binding for the migration of leukocytes, making the immune cells travel more freely, and as inducer of antiviral effects

12-16. (canceled)
